SEQUENCE LISTING (4 sequences)

SEQ ID NO: 1 - protein, 876 aa, Artificial Sequence
  Synthetic (amino acid sequence of DDR1)
MGPEALSSLLLLLLVASGDADMKGHFDPAKCRYALGMQDRTIPDSDISASSSWSDSTAARHSRLESSDGDGAWCPAGSVFPKEEEYLQVDLQRLHLVALVGTQGRHAGGLGKEFSRSYRLRYSRDGRRWMGWKDRWGQEVISGNEDPEGVVLKDLGPPMVARLVRFYPRADRVMSVCLRVELYGCLWRDGLLSYTAPVGQTMYLSEAVYLNDSTYDGHTVGGLQYGGLGQLADGVVGLDDFRKSQELRVWPGYDYVGWSNHSFSSGYVEMEFEFDRLRAFQAMQVHCNNMHTLGARLPGGVECRFRRGPAMAWEGEPMRHNLGGNLGDPRARAVSVPLGGRVARFLQCRFLFAGPWLLFSEISFISDVVNNSSPALGGTFPPAPWWPPGPPPTNFSSLELEPRGQQPVAKAEGSPTAILIGCLVAIILLLLLIIALMLWRLHWRRLLSKAERRVLEEELTVHLSVPGDTILINNRPGPREPPPYQEPRPRGNPPHSAPCVPNGSAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 2 - DNA, 3840 bp, Artificial Sequence
  Synthetic (nucleotide sequence encoding DDR1)
gtcttcccctcgtgggccctgagcgggactgcagccagccccctggggcgccagctttggaggcccccgacagctgctctcgggagccgcctcccgacacccgagccccgccggcgcctcccgctcccggctcccggctcctggctccctccgcctcccccgcccctcgccccgccgccgaagaggccccgctcccgggtcggacgcctgggtctgccgggaagagcgatgagaggtgtctgaaggtggctattcactgagcgatggggttggacttgaaggaatgccaagagatgctgcccccacccccttaggcccgagggatcaggagctatgggaccagaggccctgtcatctttactgctgctgctcttggtggcaagtggagatgctgacatgaagggacattttgatcctgccaagtgccgctatgccctgggcatgcaggaccggaccatcccagacagtgacatctctgcttccagctcctggtcagattccactgccgcccgccacagcaggttggagagcagtgacggggatggggcctggtgccccgcagggtcggtgtttcccaaggaggaggagtacttgcaggtggatctacaacgactgcacctggtggctctggtgggcacccagggacggcatgccgggggcctgggcaaggagttctcccggagctaccggctgcgttactcccgggatggtcgccgctggatgggctggaaggaccgctggggtcaggaggtgatctcaggcaatgaggaccctgagggagtggtgctgaaggaccttgggccccccatggttgcccgactggttcgcttctacccccgggctgaccgggtcatgagcgtctgtctgcgggtagagctctatggctgcctctggagggatggactcctgtcttacaccgcccctgtggggcagacaatgtatttatctgaggccgtgtacctcaacgactccacctatgacggacataccgtgggcggactgcagtatgggggtctgggccagctggcagatggtgtggtggggctggatgactttaggaagagtcaggagctgcgggtctggccaggctatgactatgtgggatggagcaaccacagcttctccagtggctatgtggagatggagtttgagtttgaccggctgagggccttccaggctatgcaggtccactgtaacaacatgcacacgctgggagcccgtctgcctggcggggtggaatgtcgcttccggcgtggccctgccatggcctgggagggggagcccatgcgccacaacctagggggcaacctgggggaccccagagcccgggctgtctcagtgccccttggcggccgtgtggctcgctttctgcagtgccgcttcctctttgcggggccctggttactcttcagcgaaatctccttcatctctgatgtggtgaacaattcctctccggcactgggaggcaccttcccgccagccccctggtggccgcctggcccacctcccaccaacttcagcagcttggagctggagcccagaggccagcagcccgtggccaaggccgaggggagcccgaccgccatcctcatcggctgcctggtggccatcatcctgctcctgctgctcatcattgccctcatgctctggcggctgcactggcgcaggctcctcagcaaggctgaacggagggtgttggaagaggagctgacggttcacctctctgtccctggggacactatcctcatcaacaaccgcccaggtcctagagagccacccccgtaccaggagccccggcctcgtgggaatccgccccactccgctccctgtgtccccaatggctctgcctacagtggggactatatggagcctgagaagccaggcgccccgcttctgcccccacctccccagaacagcgtcccccattatgccgaggctgacattgttaccctgcagggcgtcaccgggggcaacacctatgctgtgcctgcactgcccccaggggcagtcggggatgggccccccagagtggatttccctcgatctcgactccgcttcaaggagaagcttggcgagggccagtttggggaggtgcacctgtgtgaggtcgacagccctcaagatctggttagtcttgatttcccccttaatgtgcgtaagggacaccctttgctggtagctgtcaagatcttacggccagatgccaccaagaatgccaggaatgatttcctgaaagaggtgaagatcatgtcgaggctcaaggacccaaacatcattcggctgctgggcgtgtgtgtgcaggacgaccccctctgcatgattactgactacatggagaacggcgacctcaaccagttcctcagtgcccaccagctggaggacaaggcagccgagggggcccctggggacgggcaggctgcgcaggggcccaccatcagctacccaatgctgctgcatgtggcagcccagatcgcctccggcatgcgctatctggccacactcaactttgtacatcgggacctggccacgcggaactgcctagttggggaaaatttcaccatcaaaatcgcagactttggcatgagccggaacctctatgctggggactattaccgtgtgcagggccgggcagtgctgcccatccgctggatggcctgggagtgcatcctcatggggaagttcacgactgcgagtgacgtgtgggcctttggtgtgaccctgtgggaggtgctgatgctctgtagggcccagccctttgggcagctcaccgacgagcaggtcatcgagaacgcgggggagttcttccgggaccagggccggcaggtgtacctgtcccggccgcctgcctgcccgcagggcctatatgagctgatgcttcggtgctggagccgggagtctgagcagcgaccacccttttcccagctgcatcggttcctggcagaggatgcactcaacacggtgtgaatcacacatccagctgcccctccctcagggagcgatccaggggaagccagtgacactaaaacaagaggacacaatggcacctctgcccttcccctcccgacagcccatcacctctaatagaggcagtgagactgcaggtgggctgggcccacccagggagctgatgccccttctccccttcctggacacactctcatgtccccttcctgttcttccttcctagaagcccctgtcgcccacccagctggtcctgtggatgggatcctctccaccctcctctagccatcccttggggaagggtggggagaaatataggatagacactggacatggcccattggagcacctgggccccactggacaacactgattcctggagaggtggctgcgcccccagcttctctctccctgtcacacactggaccccactggctgagaatctgggggtgaggaggacaagaaggagaggaaaatgtttccttgtgcctgctcctgtacttgtcctcagcttgggcttcttcctcctccatcacctgaaacactggacctgggggtagccccgccccagccctcagtcacccccacttcccacttgcagtcttgtagctagaacttctctaagcctatacgtttctgtggagtaaatattgggattggggggaaagagggagcaacggcccatagccttggggttggacatctctagtgtagctgccacattgatttttctataatcacttggggtttgtacatttttggggggagagacacagatttttacactaatatatggacctagcttgaggcaattttaatcccctgcactaggcaggtaataataaaggttgagttttccacaaaaaaaaaaaaaaaaaa

SEQ ID NO: 3 - protein, 314 aa, Artificial Sequence
  Synthetic (amino acid sequence of EpCAM)
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 4 - DNA, 1731 bp, Artificial Sequence
  Synthetic (nucleotide sequence encoding EpCAM)
aactgcagcgccggggctgggggaggggagcctactcactcccccaactcccgggcggtgactcatcaacgagcaccagcggccagaggtgagcagtcccgggaaggggccgagaggcggggccgccaggtcgggcaggtgtgcgctccgccccgccgcgcgcacagagcgctagtccttcggcgagcgagcaccttcgacgcggtccggggaccccctcgtcgctgtcctcccgacgcggacccgcgtgccccaggcctcgcgctgcccggccggctcctcgtgtcccactcccggcgcacgccctcccgcgagtcccgggcccctcccgcgcccctcttctcggcgcgcgcgcagcatggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccatgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatagcagttgttgctggaattgttgtgctggttatttccagaaagaagagaatggcaaagtatgagaaggctgagataaaggagatgggtgagatgcatagggaactcaatgcataactatataatttgaagattatagaagaagggaaatagcaaatggacacaaattacaaatgtgtgtgcgtgggacgaagacatctttgaaggtcatgagtttgttagtttaacatcatatatttgtaatagtgaaacctgtactcaaaatataagcagcttgaaactggctttaccaatcttgaaatttgaccacaagtgtcttatatatgcagatctaatgtaaaatccagaacttggactccatcgttaaaattatttatgtgtaacattcaaatgtgtgcattaaatatgcttccacagtaaaatctgaaaaactgatttgtgattgaaagctgcctttctatttacttgagtcttgtacatacatacttttttatgagctatgaaataaaacattttaaactgaatttcttaaaaaaaaaaaaaaa

(19) United States
(12) Patent Application Publication
(10) Pub. No.: US 2015/0064721 A1
(43) Pub. Date: Mar. 5, 2015

(54) COMPOSITION AND KIT FOR SEPARATING CANCER CELL, AND METHOD OF SEPARATING CANCER CELL BY USING THE COMPOSITION AND KIT

(71) Applicants: Ajou University Industry Cooperation Foundation, Suwon-si (KR); Samsung Electronics Co., Ltd., Suwon-si (KR)

(72) Inventors: Yeon-jeong KIM, Yongin-si (KR); Kyung-yeon Han, Seoul (KR); You-Sun Kim, Suwon-si (KR); Hui-sung Moon, Seoul (KR); Dong-hyun Park, Chuncheon-si (KR); Jong-myeon Park, Incheon (KR)

(73) Assignees: Ajou University Industry Cooperation Foundation, Suwon-si (KR); Samsung Electronics Co., Ltd., Suwon-si (KR)

(21) Appl. No.: 14/252,284

(22) Filed: Apr. 14, 2014

(30) Foreign Application Priority Data
Sep. 3, 2013 (KR) 10-2013-0105695

(51) Int. Cl. G01N 33/574; C07K 16/30; C07K 16/40

(52) U.S. Cl. CPC G01N 33/57492; C07K 16/40; C07K 16/30; G01N 33/57415; G01N 33/57423; G01N 33/57434; G01N 2333/70596
USPC 435/7.23; 530/387.9; 530/387.3

(57) ABSTRACT

Provided is a composition and a kit for separating a cancer stem cell or a circulating tumor cell, a method of separating a cancer stem cell or a circulating tumor cell in a biological sample, and a method for diagnosing metastatic cancer.

### RELATED APPLICATION

This application claims the benefit of Korean Patent Application No. 10-2013-0105695, filed on Sep. 3, 2013, in the Korean Intellectual Property Office, the disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: 18,314 bytes ASCII (Text) file named “715871_ST25.TXT,” created Apr. 7, 2014.

### BACKGROUND

1. Field

The present invention relates to compositions and kits for separating cancer stem cells or circulating tumor cells, methods for separating cancer stem cells or circulating tumor cells from biological samples using the compositions and kits, and methods for diagnosing metastatic cancer using the compositions and kits.

2. Description of the Related Art

Tumor metastasis, which occurs in patients with solid cancer, is a process that involves the release of some tumor cells and migration of these cells to other parts of the body via blood vessels. Tumor metastasis is a major cancer-related cause of death.

Circulating tumor cells (CTCs) are rare tumor cells present in blood due to tumor invasion and thus circulate in the body. CTCs are known to be involved in the metastasis and recurrence of cancer. It has been suggested that circulating tumor cells are likely to contain cancer stem cells.

The CTCs or disseminated tumor cells that are present in other organs of a patient may be separated or detected to enable an accurate and a quick diagnosis of metastatic cancer. In addition, the efficacy of a patient's drug treatment regimen may be enhanced by administration of anticancer drugs selected specifically for the patient based on the detection and/or separation (i.e., determining the presence or absence) of CTCs in a sample provided by the patient. Thus, the detection or separation of CTCs in a sample of a patient can enable a physician to provide a personalized drug regimen tailored to molecular properties of CTCs.

Since CTCs are present in small amounts in the blood and are fragile, it is difficult to accurately detect and count the number of CTCs present in a sample. Therefore, a need remains to develop methods for efficiently separating and detecting the CTCs or the cancer stem cells present in patients, as well as diagnosing metastatic cancer with higher accuracy and sensitivity.

### SUMMARY

Provided are compositions and kits for separating cancer stem cells or circulating tumor cells comprising a substance that specifically binds discoidin domain receptor 1 (DDR 1) or a fragment thereof.

Additionally, provided is a method for separating at least one of a cancer stem cell and a circulating tumor cell from a biological sample, the method comprising:

incubating a biological sample from a subject with a substance that specifically binds to DDR 1 or a fragment thereof to form a reaction mixture, whereby the substance binds to DDR 1 or a fragment thereof on a cancer stem cell or circulating tumor cell in the biological sample; and separating a cancer stem cell or circulating tumor cell bound to the substance that binds DDR 1 or fragment thereof from the reaction mixture. The method can be used in the diagnosis or prognosis of metastatic cancer, wherein the detection of a cancer stem cell or circulating tumor cell in the biological sample is indicative of metastatic cancer, or a risk of developing metastatic cancer, in the subject.

Related compositions, kits, and methods are also provided.

### BRIEF DESCRIPTION OF THE DRAWINGS

These and/or other aspects will become apparent and more readily appreciated from the following description of the embodiments, taken in conjunction with the accompanying drawings in which:

FIG. 1 is a graph showing the binding efficacy (Y-axis) of beads comprising anti-EpCAM antibodies to cancer cell lines MCF-7, MDA-MB231, and a mixture thereof;

FIG. 2 is a table displaying the microarray results analyzing cancer cells that have induced epithelial-mesenchymal transition;

FIGS. 3A, 3B, and 3C are images showing western blotting results of breast cancer cells (3A), lung cancer cells (3B), and prostate cancer cells (3C) induced epithelial-mesenchymal transition, respectively; and

FIGS. 4A and 4B are graphs displaying the binding efficacy (Y-axis, FIG. 4A), of beads comprising anti-EpCAM antibodies and/or anti-DDR antibodies; and the recovery rate of breast cancer cells (Y-axis, FIG. 4B), both before and after epithelial-mesenchymal transition, respectively.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

According to an aspect of the present invention, provided is a composition or kit for separating a cancer stem cell or a circulating tumor cell from a sample, the composition or kit comprising a substance that specifically binds to discoidin domain receptor (DDR) 1 or a fragment thereof.

DDR 1 or a fragment thereof may be DDR 1 or a fragment thereof of a human or a mouse. DDR 1 is a protein tyrosine kinase comprising a discoidin I motif in an extracellular domain, a long cytoplasmic juxtamembrane (JM) region, and a kinase domain that is approximately 45% identical to that of nerve growth factor (NGF) receptor. DDR 1 is also known as CD167a. DDR 1 is a receptor tyrosine kinase activated by various types of collagen, and plays a role in cell adhesion, migration, survival, and proliferation. DDR 1 may comprise a polypeptide having an amino acid sequence (SEQ ID NO: 1) of GenBank Accession No. NP_{—}001945. DDR 1 may comprise a polypeptide encoded by a nucleotide sequence (SEQ ID NO: 2) of GenBank Accession No. NM_{—}001954. Other DDR 1 splice variants produced by alternative splicing of DDR 1 genes may be known in the art.

As used herein, a fragment of DDR 1 refers to a polypeptide having a continuous amino acid sequence of DDR 1 less than the full length sequence of DDR 1, for example, about 5 or more continuous amino acids of DDR 1, such as about 10 or more continuous amino acids, or about 20 or more continuous amino acids of DDR 1.

The substance that specifically binds to DDR 1 or a fragment thereof may be an anti-DDR 1 antibody or an antigen-binding fragment (i.e., antibody fragment) thereof, an aptamer, collagen, or a combination thereof. The antibody may be a monoclonal antibody or a polyclonal antibody, and may be also a full-length antibody or an antigen-binding fragment thereof. The antigen-binding fragment may comprise an antigen-binding site. For example, the antigen-binding fragment may be selected from the group consisting of a single-chain variable fragment (scFv), a (scFv)₂ fragment, a Fab fragment, a Fab′ fragment, a F(ab′)₂ fragment, and a combination thereof. The antibody or an antigen-binding fragment thereof, or other substance that specifically binds DDR 1 or fragment thereof, may be labeled with a molecule that facilitates detection or isolation, e.g., radionuclides, fluorescent tags (e.g., fluorescors), dyes, or enzymes. The aptamer may comprise oligonucleic acid or peptide that binds to a specific target molecule. Collagen, as a protein found in bones, skin, blood vessels, teeth, muscles, etc. of animals, is the main component of connective tissue. Collagen may comprise Collagen I, Collagen II, Collagen III, Collagen IV, or Collagen V. For example, DDR 1 may be bound to Collagen I, Collagen III, Collagen IV, or Collagen V.

The cancer stem cell may have characteristics associated with a normal stem cell, for example, self-renewal and differentiation abilities, and may be found within a tumor or hematological cancer.

The circulating tumor cell (CTC) is a rare tumor cell that enters the blood through tumor invasion and, thus, circulates in the body. CTCs are known to be involved in the metastasis and recurrence of cancer.

The cancer stem cells or the CTCs may have undergone epithelial-mesenchymal transition. The epithelial-mesenchymal transition is a process by which epithelial cells lose their cell polarity and cell-to-cell adhesion abilities, and in the process gain migratory and invasive properties, thus becoming mesenchymal cells. The epithelial-mesenchymal transition may be utilized for numerous, often necessary developmental processes including mesoderm formation and neural tube formation, and has been shown to occur in wound healing, organ fibrosis, and in the initiation of metastasis in cancer progression.

The composition or kit may further comprise a substance that specifically binds to epithelial cell adhesion molecule (EpCAM) or a fragment thereof. EpCAM is a transmembrane glycoprotein mediating Ca²⁺-dependent homotypic cell-to-cell adhesion in epithelia, and appears to play a role in cell signal transduction, migration, proliferation, and differentiation. In some embodiments, EpCAM may be human or mouse EpCAM. For example, EpCAM may comprise a polypeptide having an amino acid sequence (SEQ ID NO: 3) of GenBank Accession No. NP_{—}002345. The EpCAM may comprise a polypeptide encoded by a nucleotide sequence (SEC) ID NO: 4) of GenBank Accession No. NM_{—}002354, The substance that specifically binds to the EpCAM or a fragment thereof may be an anti-EpCAM antibody or an antigen-binding fragment thereof. The other characteristics of the anti-EpCAM antibody or antigen-binding fragment may be as described with respect to the anti-DDR 1 antibody or antibody fragment.

The composition or kit may further comprise a substance needed to detect the cancer stem cells or the CTCs in a sample, or such cells that have been separated from a sample. The detecting substance may comprise an antibody or an antigen-binding fragment thereof, a cell-staining reagent, or any other suitable detection agent.

The kit may further comprise a solid support. The solid support may comprise a globular or bead shape, a multi-sided polygon shape, a plate shape, a linear shape, or any combination thereof. The solid support may comprise polystyrene, polypropylene, melamine, magnetic particles, or any combination thereof.

According to another aspect of the present invention, there is provided a method for separating a cancer stem cell or a circulating tumor cell from a biological sample, the method comprising: incubating a biological sample from a subject with a substance that specifically binds to DDR 1 or a fragment thereof to form a reaction mixture, whereby the substance binds to DDR 1 or a fragment thereof on the cancer stem cell or the circulating tumor cell in the biological sample; and separating at least one of a cancer stem cell and a circulating tumor cell from the reaction mixture.

DDR 1 or a fragment thereof, the cancer stem cells, and the CTCs are as previously described with respect to the composition and kit of the invention, above.

The subject may be a mammal (e.g., a human),

The biological sample may comprise any sample obtained from the subject including, for instance, blood, plasma, bone marrow fluid, lymph fluid, saliva, lachrymal fluid, urine, mucous fluid, amniotic fluid, or any combination thereof.

The substance that specifically binds to DDR 1 or a fragment thereof may comprise an anti-DDR 1 antibody or an antigen-binding fragment thereof, an aptamer, or collagen. The anti-DDR 1 antibody or an antigen-binding fragment thereof, aptamer, and collagen are defined as previously described, above. The anti-DDR 1 antibody or an antigen-binding fragment thereof, or other substance that specifically binds to DDR 1 or fragment thereof, may be immobilized on a support. The support may comprise a solid support, and the solid support may comprise a globular or bead shape, a multi-sided polygon shape, a plate shape, a linear shape, or any combination thereof. The solid support may comprise polystyrene, polypropylene, melamine, magnetic particles, or any combination thereof.

The incubation may be performed in vitro at room temperature or a temperature of 4° C. The incubation may be performed for a period of time sufficient for a target cell (i.e., cancer stem cells and/or CTCs) and the substance that specifically binds to DDR 1 or a fragment thereof to be sufficiently bound. For example, the incubation may be performed for about 10 minutes, e.g., about 1 hour, about 5 hours, about 12 hours or about 24 hours.

The cancer stem cell or the circulating tumor cell may be separated from the reaction mixture by any suitable technique. The reaction mixture may comprise a complex in which a cancer stem cell or a circulating tumor cell is bound to a substance that specifically binds to DDR 1 or a fragment thereof. The complex of the cancer stem cell or circulating tumor cell and substance that binds to DDR 1 or fragment thereof may be separated from the biological sample. Alternatively, or in addition, the cancer stem cell or the circulating tumor cell may be separated from the complex (e.g., the cancer stem cell or circulating tumor cell may be separated from the substance that binds DDR 1 or fragment thereof). The separation may be conducted by microfiltration, centrifugation, micro flow, immunostaining, immunoprecipitation, enzyme-linked immunosorbent assay (ELISA), flow cytometry, fluorescence-activated cell sorting (FACS), or any combination thereof. Some embodiments may also comprise a step of washing the complex or complexes formed during the incubation step.

Some embodiments may also include the step of incubating the biological sample provided by a subject with a second substance, wherein the second substance specifically binds EpCAM or a fragment thereof, whereby the second substance binds to EpCAM on at least one of a cancer stern cell and a circulating tumor cell in the sample. The biological sample can be incubated with the second substance that binds EpCAM before, after, or simultaneously with the substance that binds to DDR 1 or fragment thereof.

All other aspects of the method are as described with respect to the composition, kit, and other methods of the invention.

Some embodiments may further include the step of detecting at least one of a cancer stem cell and a CTC (e.g., a complex of the cancer stern cell or CTC bound to a substance that specifically binds to DDR 1 or fragment thereof) before or after separation from the reaction mixture. The detection step may be conducted by electron microscopic observation, microfiltration, centrifugation, micro flow, immunostaining, immunoprecipitation, ELISA, flow cytometry, FACS, or any combination thereof. The detection step may be facilitated by a detectable label on the substance that specifically binds DDR 1 or a fragment thereof. Any detectable label can be used, for instance, radionucleotides, fluorescent Lags (e.g., fluorescors), dyes, or enzymes.

According to another aspect of the present invention, there is provided a method for diagnosing metastatic cancer, the method comprising: incubating a biological sample from a subject with a substance that specifically binds to DDR 1 or a fragment thereof to form a reaction mixture, whereby the substance binds to DDR 1 or a fragment thereof on at least one of a cancer stem cell and a circulating tumor cell in the biological sample; detecting a cancer stem cell or circulating tumor cell by separating a cancer stem cell or circulating tumor cell bound to the substance that specifically binds DDR 1 from the reaction mixture; and

determining that a subject has metastatic cancer or is likely to develop metastatic cancer based on the detection of a cancer stem cell or circulating tumor cell. All other aspects of the method are as previously described with respect to the composition, kit, and other methods of the invention.

Metastatic cancer is a cancer that involves the spreading of a cancer from an onset organ to another organ that includes a lymph node. Thus, the cancer existing in the onset organ is known as a primary cancer. Depending on the tissue a primary cancer spreads to, metastasis may comprise brain metastasis, bone metastasis, liver metastasis, or lung metastasis. Metastatic cancer may comprise a malignant tumor, and the malignant tumor may comprise a brain spinal tumor, head and neck cancer, lung cancer, breast cancer, thymoma, mesothelioma, esophageal cancer, stomach cancer, colorectal cancer, liver cancer, pancreatic cancer, biliary tract cancer, renal cancer, bladder cancer, prostate cancer, testicular cancer, germ cell tumor, ovarian cancer, cervical cancer, endometrial cancer, lymphoma, acute leukemia, chronic leukemia, multiple myeloma, sarcoma, malignant melanoma, skin cancer, or any combination thereof.

According to another aspect of the present invention, provided is a method for providing information needed for diagnosis of metastatic cancer, the method comprising:

incubating a biological sample provided by a subject with a substance that specifically binds to DDR 1 or a fragment thereof to form a reaction mixture, whereby the substance binds to DDR 1 or fragment thereof on the surface of at least one of a cancer stern cell and a circulating tumor cell in the biological sample; detecting a cancer stem cell or circulating tumor cell by separating at least one of a cancer stem cell and a circulating tumor cell bound to the substance that binds DDR 1 or fragment thereof from the reaction mixture; and

determining that a subject already has metastatic cancer or is likely to have metastatic cancer based on the detection of the cancer stern cell or the circulating tumor cell.

Thus, the compositions, kits, and methods provided herein offer means for efficiently and accurately separating and/or detecting cancer stem cells and/or CTCs with high degrees of sensitivity.

One or more embodiments of the present invention will now be described in detail with reference to the following examples. However, these examples are not intended to limit the scope of the one or more embodiments of the present invention.

### EXAMPLE 1

### Confirmation of the Binding Efficacy of Beads Conjugated with Anti-EpCAM Antibodies to Breast Cancer Cell Lines

1-1. Preparation of Beads Conjugated with Anti-EpCAM Antibodies Bound Thereto

COOH melamine beads (Sigma) having a diameter of 1 or 3 μm were treated with EDC(N-hydroxysuccinimide)/NHS(1-ethyl-3-[3-dimethylaminopropyl]carbodiimide hydrochloride) and then the treated beads were added to a PBS buffer solution, 250 μg/ml of anti-EpCAM antibodies (R&D system) or anti-DDR 1 antibodies (Abcam) were added to the resulting solution, and the resulting solution was slowly shaken and incubated at room temperature for 2 hours. Next, the incubated solution was washed with a PBS buffer solution at room temperature, and then incubated in 2% (w/v) of bovine serum albumin (BSA) solution for 1 hour, thereby completing the preparation of beads to which anti-EpCAM antibodies or anti-DDR 1 antibodies were conjugated.

1-2. Confirmation of the Binding efficacy of Beads Conjugated with Anti-EpCAM Antibodies to Breast Cancer Cell Lines MCF7 or MDA-MB231

The binding efficacy of the beads conjugated with anti-EpCAM antibodies bound thereto to breast cancer cell lines MCF7 (purchased from American Type Culture Collection (ATCC)) with a high expression level of EpCAM, or to breast cancer cell lines MDA-MB231 (ATCC) with a low expression level of EpCAM was confirmed in this experiment.

1×10⁵ cells of breast cancer cell lines MCF7 and 1×10⁵ cells of breast cancer cell lines MDA-BM231 were each incubated with eosin (Sigma) and fluorescein (Sigma), and then the MCF7 and MDA-BM231 were each stained with a red fluorescence material and a green fluorescence material.

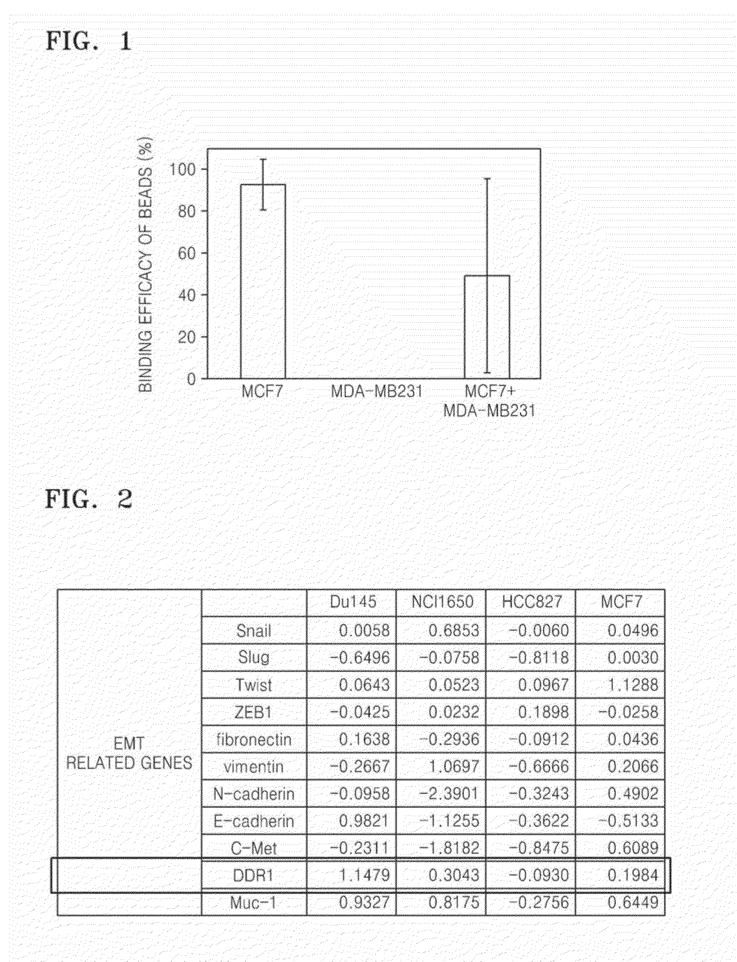

The stained breast cancer cell lines MCF7 and MDA-BM231 were suspended in a DMEM medium, and 20 μm of the beads conjugated with anti-EpCAM antibodies bound thereto, which were prepared according to Example 1-1, was added to reaction mixture and incubated at room temperature for 1 hour. Then, 30 images showing the binding of the beads to the breast cancer cells were obtained by using a fluorescence microscope (Olympus IX-81), and the bead-bound areas on the total cell surface were calculated by using the Image J (NIH) program so as to yield the binding efficacy of the beads to the breast cancer cells. The red fluorescent MCF7 had a high expression level of EpCAM, whereas the green fluorescent MDA-MB231 had low expression of EpCAM. The comparative binding efficacy of the anti-EpCAM beads to the MCF7 or MDA-MB231 was confirmed by the obtained images. A graph displaying the binding efficacy of the beads to the MCF7, MDA-MB231, and a mixture thereof is shown in FIG. 1.

As seen in FIG. 1, it was confirmed that the binding efficacy of the beads conjugated with anti-EpCAM antibodies to the MCF7 was about 93%, whereas the binding efficacy of the beads conjugated with anti-EpCAM antibodies bound thereto to the MDA-MB231 was about 0%. Thus, these results demonstrate that detection or separation of cells from heterogeneous cell populations that include cells with low expression level of EpCAM requires an antibody that specifically binds to a marker other than EpCAM y.

### Example 2

### Confirmation of DDR 1 Expression in Cancer Cell Lines Undergone Epithelial-Mesenchymal Transition

2-1. Artificially Induced Epithelial-Mesenchymal Transition in Breast Cancer Cell Lines, Lung Cancer Cell Lines, or Prostate Cancer Cell Lines

Blood cells HL60, THP-1, and U937, lung cancer cell lines HCC827 and H1650, and prostate cancer cell lines Du145 were cultured in an RPMI1640 medium containing 10% (v/v) FBS, and the breast cell lines MCF7 were cultured in a DMEM medium containing 10% (v/v) FBS. To induce epithelial-mesenchymal transition in the breast cancer cell lines MCF7, the lung cancer cell lines HCC827 and H1650, and the prostate cancer cell lines DU145 and PC3, a mammosphere culture method described below was used instead of the existing attachment culture method culturing in a DMEM with 10% FBS. A medium containing DMEM-F12, 1×B27, 20 ng/ml FGF, 20 ng/ml EGF, and 5 μg/ml insulin was used as a culture medium, and the cancer cells (2×10⁵ cells/ml) were inoculated in a 100 mm dish and then cultured at a temperature of 37° C. for 24 hours to 3 weeks.

2-2. Confirmation of Induction of Epithelial-Mesenchymal Transition and DDR I Expression in Cell Lines that had Undergone Epithelial-Mesenchymal Transition

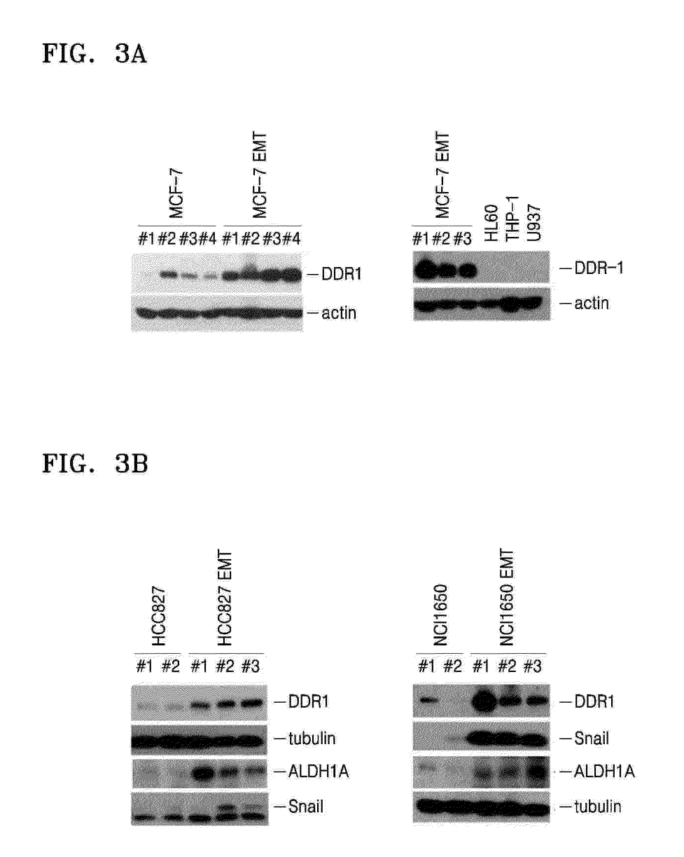

After mRNA was separated from the cells which were cultured according to Example 2-1, mRNA was subjected to reverse transcription and microarray analysis. 500 ng of total RNA in each sample was converted into cDNA by using an ILLUMINA®TOTALPREP™ RNA amplification kit (Ambion Inc), and then the cDNA was hybridized to a human HT12-v4 IlluminaBeadchip gene expression array (Illumina), and scanned and analyzed fluorescent signals by using an Illumina Bead Array Reader (Illumina). The resulting microarray data are shown in FIG. 2.

As seen in FIG. 2, it was confirmed that the expression of a snail gene, a twist gene, a fibronectin gene, and a DDR 1 gene increased post-inoculation. It was confirmed that the cell lines had undergone epithelial-mesenchymal transition by confirming the snail gene, the twist gene, and the fibronectin gene, which are markers known to have increased expression when epithelial-mesenchymal transition was induced. The results in FIG. 2 also confirmed that the expression of DDR 1 genes have increased in the cells that had undergone epithelial-mesenchymal transition.

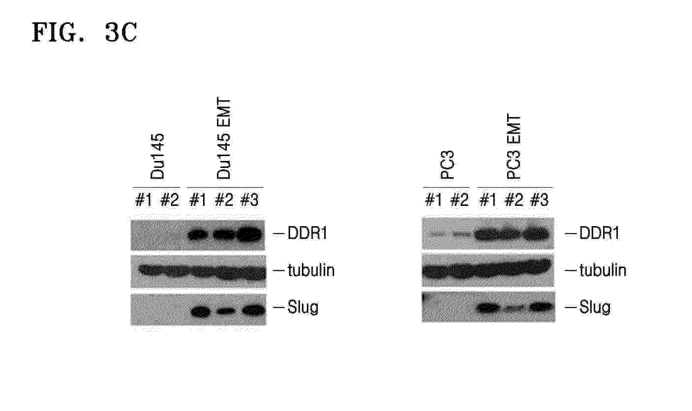

After proteins were separated from the cells which were cultured according to Example 2-1, the proteins were performed by electrophoresis and western blotting. An electrophoresized gel was transferred to film, and then western blotting was performed using an anti-SNAIL antibody (Abcam), an anti-ALDH1 antibody (Abcam), an anti-DDR 1 antibody (Abcam), an anti-tubulin antibody (Abcam), an anti-Slug antibody (Abcam), and an anti-actin antibody (cell signaling). The western blotting results are shown in FIGS. 3A through 3C.

As shown in FIGS. 3A through 3C, it was confirmed that the amount of Snail protein, ALDH1 protein, and DDR 1 protein increased in the lung cancer cells in which epithelial-mesenchymal transition was induced, and the amount of DDR 1 increased in the breast cancer cells and the prostate cancer cells in which epithelial-mesenchymal transition was induced. It was confirmed that the amount of DDR 1 protein increased in the cancer cells that had undergone epithelial-mesenchymal transition and cancer stem cells by confirming the increase in the amount of Snail protein, which is known as an epithelial-mesenchymal transition marker, and the amount of ALDH1, which is a protein as a cancer stem cell marker.

As seen in FIGS. 2, and 3A through 3C, it was confirmed that the DDR 1 protein may be used as a marker for separating cancer cells that have undergone epithelial-mesenchymal transition or for cancer stem cells due to the up regulation of DDR 1 in cells that underwent epithelial-mesenchymal transitions.

### Example 3

### Separation of Breast Cancer Cells that have Undergone Epithelial-Mesenchymal Transition in the Blood Using Beads Bound to Anti-DDR 1 Antibodies

3-1. Preparation of Beads with Anti-DDR 1 Antibodies Bound Thereto

The beads with anti-DDR 1 antibodies bound thereto were prepared in the same manner as in Example 1-1.

3-2. Separation of Breast Cancer Cells that have Undergone Epithelial-Mesenchymal Transition in the Blood

5 ml of the blood of healthy people was mixed with 100 breast cancer cells that had not undergone epithelial-mesenchymal transition, or 100 breast cancer cells that had undergone epithelial-mesenchymal transition and prepared according to Example 2-1.

The beads with an anti-EpCAM antibody bound thereto which were prepared according to Example 1-1, and the beads with an anti-DDR 1 antibody bound thereto which were prepared according to Example 3-1.

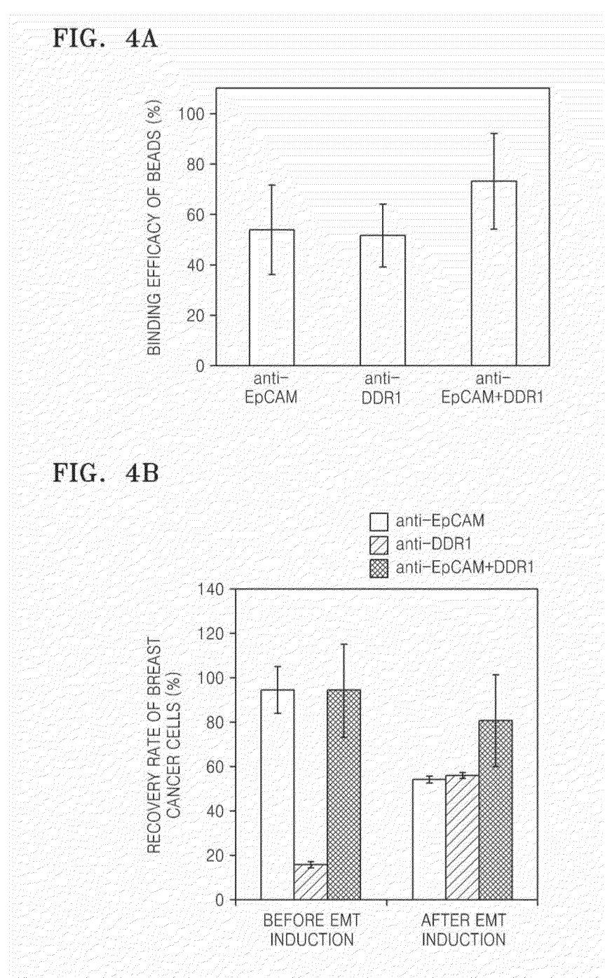

The blood mixed with breast cancer cells was divided into three groups, and 100 breast cancer cells were added thereto. Then, each group had 1×10⁷ beads with anti-EpCAM antibodies bound thereto, 1×10 beads with anti-DDR 1 antibodies bound thereto, and a mixture of 0.5×10⁷ beads with anti-EpCAM antibodies bound thereto and 0.5×10⁷ beads with anti-EpCAM antibodies bound thereto. The three reaction mixtures were incubated at room temperature for 1 hour, and then washed out with PBS buffer. As described in Example 1-2, in each group, the binding efficacy of the beads as well as the recovery rate of the breast cancer cells were calculated in each group. The result of the binding efficacy is shown in FIG. 4A, and the result of the recovery rate is shown in FIG. 4B.

As seen in FIG. 4A, it was confirmed that the breast cancer cells that had undergone epithelial-mesenchymal transition show a binding efficacy of about 50% or more with the beads with anti-EpCAM antibodies bound thereto, or with the beads with anti-DDR 1 antibodies bound thereto. In addition, it was confirmed that the breast cancer cells that had undergone epithelial-mesenchymal transition show a binding efficacy of about 70% with the mixture of the beads with anti-EpCAM antibodies and anti-DDR 1 antibodies bound thereto. As seen in FIG. 4B, the breast cancer cells that had not undergone epithelial-mesenchymal transition show low recovery rates, but the breast cancer cells in which epithelial-mesenchymal transition was induced show increased recovery rates due to the addition of the beads with anti-DDR 1 antibodies bound thereto.

Both FIGS. 4A and 4B, confirmed that, in the case of simultaneous use of anti-EpCAM antibodies and anti-DDR 1 antibodies, the binding efficacy of the beads as well as the recovery rate of the breast cancer cells in the blood are significantly increased, demonstrating the possibility and potential for the use of DDR 1 as a suitable marker for the separation of cancer cells.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (Le., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

What is claimed is:

1. A composition comprising a substance that specifically binds to discoidin domain receptor (DDR) 1 or a fragment thereof on the surface of a cancer stem cell or circulating tumor cell.

2. The composition of claim 1, wherein the cancer stem cell or the circulating tumor cell has undergone epithelial-mesenchymal transition.

3. The composition of claim 1, wherein the substance that specifically binds to DDR 1 is an anti-DDR 1 antibody or an antigen-binding fragment thereof, an aptamer, collagen, or any combination thereof.

4. The composition of claim 3, wherein the antibody is a monoclonal antibody or a polyclonal antibody.

5. The composition of claim 3, wherein the antigen-binding fragment is an scFv fragment, a (scFv)₂ fragment, a Fab fragment, a Fab′ fragment, a F(ab′)₂ fragment, or any combination thereof.

6. The composition of claim 1, further comprising a second substance that specifically binds to epithelial cell adhesion molecule (EpCAM) or a fragment thereof.

7. The composition of claim 6, wherein the second substance is an anti-EpCAM antibody or an antigen-binding fragment thereof, an aptamer, collagen, or any combination thereof.

8. The composition of claim 1, wherein the composition comprises:
(a) a cancer stem cell or circulating tumor cell; and
(b) a substance bound to DDR 1 or a fragment thereof on the surface of the cancer stem cell or circulating tumor cell, wherein the substance is an anti-DDR 1 antibody or an antigen-binding fragment thereof, an aptamer specific to DDR 1, or collagen with a DDR 1 binding site;
wherein the substance bound to DDR 1 or a fragment thereof on the surface of the cancer stem cell or circulating tumor cell is optionally immobilized on a solid support.

9. A method of separating at least one of a cancer stern cell and a circulating tumor cell from a biological sample, the method comprising:
incubating a biological sample from a subject with a substance that specifically binds to DDR 1 or a fragment thereof to provide a reaction mixture, whereby the substance binds to DDR 1 or a fragment thereof on a cancer stem cell or circulating tumor cell in the biological sample; and
separating the cancer stem cell or the circulating tumor cell bound to the substance that binds DDR 1 or fragment thereof from the reaction mixture.

10. The method of claim 9, wherein the biological sample comprises blood, plasma, bone marrow fluid, lymph fluid, saliva, lachrymal fluid, urine, mucous fluid, amniotic fluid, or any combination thereof.

11. The method of claim 9, wherein the substance that specifically binds to DDR 1 or a fragment thereof is an anti-DDR 1 antibody or an antigen-binding fragment thereof, an aptamer, a collagen, or any combination thereof.

12. The method of claim 11, wherein the substance that specifically binds DDR 1 is an anti-DDR 1 antibody or antigen-binding fragment immobilized on a support.

13. The method of claim 9, further comprising incubating the biological sample with a second substance that specifically binds to EpCAM or a fragment thereof, whereby the second substance binds to EpCAM or fragment thereof on a cancer stem cell or circulating tumor cell in the biological sample.

14. A method for diagnosing metastatic cancer, the method comprising:
incubating a biological sample from a subject with a substance that specifically binds DDR 1 or a fragment thereof to form a reaction mixture, whereby the substance binds to DDR 1 or a fragment thereof on a cancer stem cell or circulating tumor cell in the biological sample;
detecting the presence of a cancer stem cell or circulating tumor cell in the biological sample by separating a cancer stem cell or circulating tumor cell bound to the substance that binds DDR 1 or fragment thereof from the reaction mixture; and
determining that a subject has metastatic cancer or is likely to have metastatic cancer based on result of the detection of a cancer stem cell or circulating tumor cell in the biological sample.

15. The method of claim 14, wherein the substance that specifically binds DDR 1 or a fragment thereof is immobilized on a solid support.

16. The method of claim 14, wherein the substance that binds DDR 1 or a fragment thereof further comprises a detectable label.
